(19) United States
(12) Patent Application Publication
(10) Pub. No.: US 2022/0104744 A1
(43) Pub. Date: Apr. 7, 2022

(54) EVALUATION DEVICE, EVALUATION METHOD, AND MEDIUM

(71) Applicant: JVCKENWOOD Corporation, Yokohama-shi (JP)

(72) Inventor: Katsuyuki Shudo, Yokohama-shi (JP)

(21) Appl. No.: 17/552,374

(22) Filed: Dec. 16, 2021

Related U.S. Application Data
(63) Continuation of application No. PCT/JP2020/024915, filed on Jun. 24, 2020.

(30) Foreign Application Priority Data
Jul. 17, 2019 (JP) 2019-132007

(51) Int. Cl. A61B 5/16; A61B 3/00; A61B 5/00

(52) U.S. Cl. CPC A61B 5/163; A61B 3/113; A61B 5/4088; A61B 3/0091

(57) ABSTRACT

An evaluation device includes a display unit; a point-of-gaze detector configured to detect a position of a point of gaze of a subject on the display unit; a display controller configured to display, on the display unit, an image containing an induction area that tends to induce visual hallucinations in the subject; a determination unit configured to, based on position data on the point of gaze, determine whether the point of gaze is staying; an area detector configured to, when the determination unit determines that the point of gaze is staying, calculate an area of staying in which the point of gaze is staying in the display unit; an acquisition unit configured to acquire a feature value representing a state of display of the area of staying that is calculated by the area setting unit; and an evaluation unit.

### CROSS-REFERENCE TO RELATED APPLICATION

This application is a Continuation of PCT International Application No. PCT/JP2020/024915 filed on Jun. 24, 2020 which claims the benefit of priority from Japanese Patent Application No. 2019-132007 filed on Jul. 17, 2019, the entire contents of both of which are incorporated herein by reference.

### BACKGROUND

### 1. Technical Field

The present disclosure relates to an evaluation device, an evaluation method, and a medium.

### 2. Description of the Related Art

In recent years, cognitive dysfunction and brain dysfunction are considered to be on the rise and it is required to find such cognitive dysfunction and brain dysfunction at early stages and quantitatively evaluate severity of symptoms. A method of asking a subject whether the subject has visual hallucinations and calling on the subject to answer when making an evaluation on, for example, dementia with Lewy bodies among such cognitive dysfunction and brain dysfunction has been disclosed (for example, refer to Japanese Laid-open Patent Publication No. 2017-217051 A).

The method described in Japanese Laid-open Patent Publication No. 2017-217051 A however depends on a subjective view of a subject and thus is with low objectivity and it is difficult to obtain accurate evaluation.

### SUMMARY

It is an object of the present disclosure to at least partially solve the problems in the conventional technology.

An evaluation device includes a display unit, a point-of-gaze detector configured to detect a position of a point of gaze of a subject on the display unit, a display controller configured to display, on the display unit, an image containing an induction area that tends to induce visual hallucinations in the subject, a determination unit configured to, based on position data on the point of gaze, determine whether the point of gaze is staying, an area detector configured to, when the determination unit determines that the point of gaze is staying, calculate an area of staying in which the point of gaze is staying in the display unit, an acquisition unit configured to acquire a feature value representing a state of display of the area of staying that is calculated by the area detector, and an evaluation unit configured to, based on the feature value that is acquired by the acquisition unit, calculate evaluation data on the subject.

An evaluation method includes detecting a position of a point of gaze of a subject on a display unit, displaying, on the display unit, an image containing an induction area that tends to induce visual hallucinations in the subject, based on position data on the point of gaze, determining whether the point of gaze is staying, when it is determined that the point of gaze is staying, detecting an area of staying in which the point of gaze is staying in the display unit, acquiring a feature value representing a state of display of the area of staying, and based on the feature value that is acquired, calculating evaluation data on the subject.

A non-transitory computer readable recording medium storing therein an evaluation program is disclosed. The evaluation program causes a computer to execute processes of detecting a position of a point of gaze of a subject on a display unit, displaying, on the display unit, an image containing an induction area that tends to induce visual hallucinations in the subject, based on position data on the point of gaze, determining whether the point of gaze is staying, when it is determined that the point of gaze is staying, detecting an area of staying in which the point of gaze is staying in the display unit, acquiring a feature value representing a state of display of the area of staying, and based on the feature value that is acquired, calculating evaluation data on the subject.

The above and other objects, features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating an example of an evaluation device according to an embodiment;

FIG. 2 is a functional block diagram illustrating an example of the evaluation device;

FIG. 3 is a table presenting an example of point-of-gaze data that is stored in a storage;

FIG. 4 is a diagram illustrating an example of an evaluation image that is displayed on a display unit;

FIG. 5 is a diagram illustrating another example of the evaluation image that is displayed on the display unit;

FIG. 6 is a flowchart illustrating an example of an evaluation method according to the embodiment;

FIG. 7 is a flowchart illustrating an example of evaluation arithmetic operations according to the embodiment; and

FIG. 8 is a flowchart illustrating the example of the arithmetic operations according to the embodiment.

### DETAILED DESCRIPTION OF THE PREFERRED EMBODIMENTS

An embodiment of an evaluation device, an evaluation method, and an evaluation program according to the disclosure will be described below based on the drawings. The embodiment does not limit the disclosure. The components in the embodiment described below include those easily replaceable by those skilled in the art or those substantially the same.

In the following description, description will be given on a positional relationship among components, setting a three-dimensional global coordinate system. A direction parallel to a first axis on a given plane is set for an X-direction, a direction parallel to a second axis on a given plane orthogonal to the first axis is set for a Y-axis direction, and a direction parallel to a third axis orthogonal to each of the first axis and the second axis is set for a Z-axis direction. The given planes include an XY-plane.

### Evaluation Device

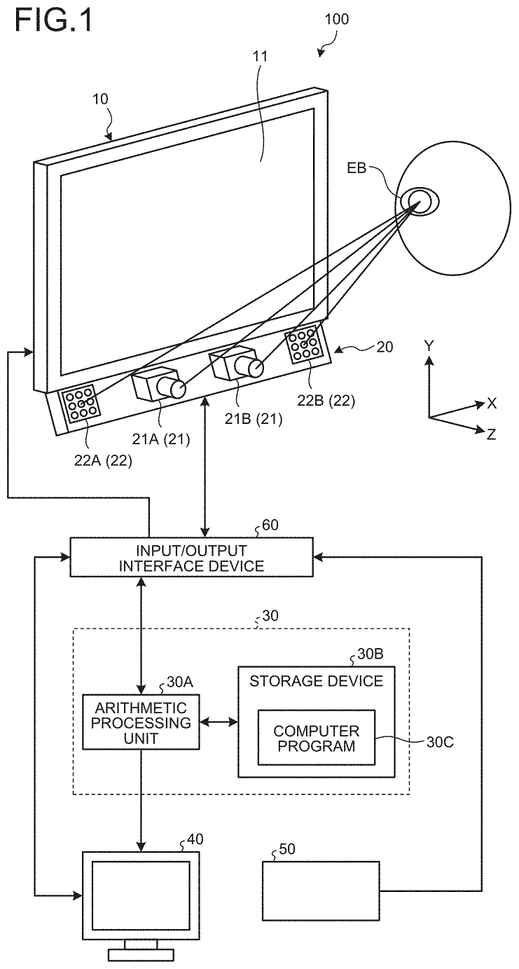

FIG. 1 is a diagram schematically illustrating an example of an evaluation device 100 according to the embodiment. The evaluation device 100 according to the embodiment detects lines of sight of a subject and, using the result of detection, makes an evaluation on cognitive dysfunction and brain dysfunction. Various devices capable of detecting lines of sight of a subject, such as, a device that detects a line of sight based on a position of a pupil of the subject and a position of a corneal reflex image or a device that detects a line of sight based on a position of an inner corner of an eye of a subject and a positon of an iris, are usable as the evaluation device 100.

As illustrated in FIG. 1, the evaluation device 100 includes a display device 10, an image acquisition device 20, a computer system 30, an output device 40, an input device 50, and an input/output interface device 60. The display device 10, the image acquisition device 20, the computer system 30, the output device 40, and the input device 50 perform data communications via the input/output interface device 60. Each of the display device 10 and the image acquisition device 20 includes a drive circuit not illustrated in the drawing.

The display device 10 includes a flat panel display, such as a liquid crystal display (LCD) or an organic electroluminescence display (OLED). In the embodiment, the display device 10 includes a display unit 11. The display unit 11 displays information, such as an image. The display unit 11 is substantially parallel to the XY-plane. The X-axis direction is a left-right direction of the display unit 11, the Y-axis direction is an up-down direction of the display unit 11, and the Z-axis direction is a depth direction orthogonal to the display unit 11. The display device 10 may be a head mounted display. In the case of a head-mounted display, such a configuration as that of the image acquisition device 20 is arranged in a head mounted module.

The image acquisition device 20 acquires image data on left and right eye balls EB of the subject and transmits the acquired image data to the computer system 30. The image acquisition device 20 includes an imaging device 21. The imaging device 21 acquires image data by capturing images of the left and right eyeballs EB of the subject. The imaging device 21 includes various cameras corresponding to a method of detecting a line of sight of the subject. For example, in the case of a system that detects a line of sight based on a position of a pupil of a subject and a position of a corneal reflex image, the imaging device 21 includes an infrared camera and includes an optical system that can transmit near-infrared light of a wavelength of 850 [nm] and an imaging element capable of receiving the near-infrared light. In the case of a system that detects a line of sight based on a position of an inner corner of an eye of a subject and a positon of an iris, the imaging device 21 includes a visible light camera. The imaging device 21 outputs a frame synchronization signal. The period of the frame synchronization signal can be set at, for example, 20 [msec]; however, the period is not limited to this. The imaging device 21 can have, for example, a configuration of a stereo camera including a first camera 21A and a second camera 21B; however, the configuration is not limited to this.

Furthermore, for example, in the case of a system that detects a line of sight based on a position of a pupil of a subject and a position of a corneal reflex image, the image acquisition device 20 includes an illuminating device 22 that illuminates the eyeballs EB of the subject. The illuminating device 22 includes a light emitting diode (LED) light source and is capable of emitting near-infrared light of, for example, a wavelength of 850 [nm]. Note that, for example, in the case of a system that detects a line-of-sight vector based on a position of an inner corner of an eye of a subject and a positon of an iris, the illuminating device 22 need not be arranged. The illuminating device 22 emits detection light in synchronization with a frame synchronization signal of the imaging device 21. The illuminating device 22 can be configured to include, for example, a first light source 22A and a second light source 22B; however, the configuration is not limited to this.

The computer system 30 have general control on operations of the evaluation device 100. The computer system 30 includes an arithmetic processing unit 30A and a storage device 30B. The arithmetic processing unit 30A includes a microprocessor, such as a central processing unit (CPU). The storage device 30B includes a memory or a storage, such as a read only memory (ROM) or a random access memory (RAM). The arithmetic processing unit 30A performs arithmetic processing according to a computer program 30C that is stored in the storage device 30B.

The output device 40 includes a display device, such as a flat panel display. Note that the output device 40 may include a printing device. The input device 50 is operated and thus generates input data. The input device 50 includes a keyboard or a mouse for computer systems. Note that the input device 50 may include a touch sensor that is arranged in the display unit of the output device 40 serving as a display device.

The evaluation device 100 according to the embodiment is a device in which the display device 10 and the computer system 30 are independent from each other. Note that the display device 10 and the computer system 30 may be integrated. For example, the evaluation device 100 may include a tablet personal computer. In this case, a display device, an image acquisition device, a computer system, an input device, an output device, etc., may be installed in the tablet personal computer.

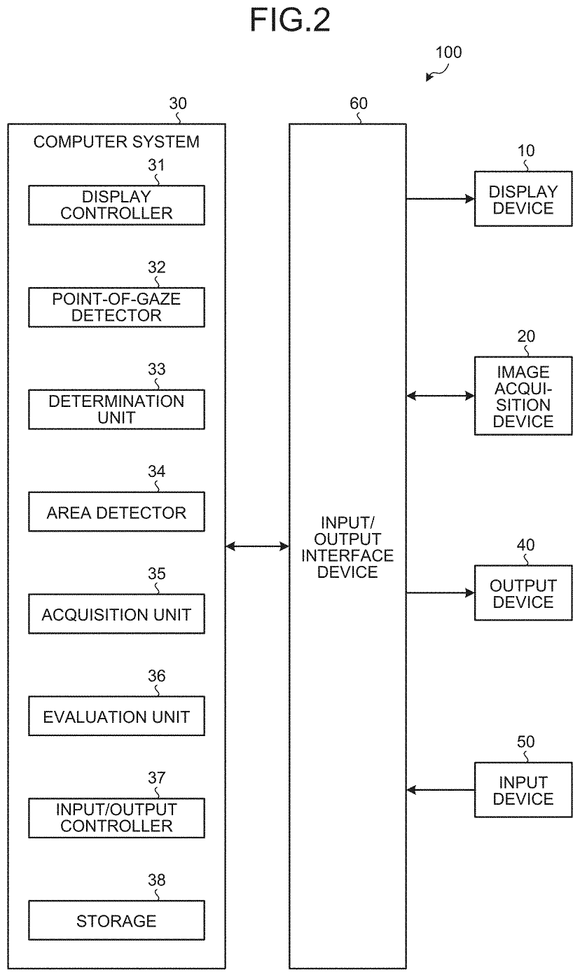

FIG. 2 is a functional block diagram illustrating an example of the evaluation device 100. As illustrated in FIG. 2, the computer system 30 includes a display controller 31, a point-of-gaze detector 32, a determination unit 33, an area detector 34, an acquisition unit 35, an evaluation unit 36, an input/output controller 37, and a storage 38. Functions of the computer system 30 are implemented by the arithmetic processing unit 30A and the storage device 30B (refer to FIG. 1). Part of the functions of the computer system 30 may be implemented outside the evaluation device 100.

The display controller 31 displays, on the display unit 11, an evaluation image (image) containing an induction area that tends to induce visual hallucinations in a subject. The evaluation image may contain, in a position different from that of the induction area, a target area serving as a target that a subject is caused to gaze at. In this case, in the evaluation image, a feature value of the induction area is lower than that of the target area. In the evaluation image, a feature value of the target area to be described below is higher than that of the surroundings. The evaluation image is, for example, a photographic image obtained by capturing an image of a scenery where a subject in the target area is present on a front side in the depth direction and a subject in the inducing area is present on a back side in the depth direction, with the subject in the target area serving as a position of a focal point. A mode of display of the evaluation image may be any one of a still image and a moving image.

The point-of-gaze detector 32 detects position data on a point of gaze of the subject. In the embodiment, based on the image data on the left and right eyeballs EB of the subject that is acquired by the image acquisition device 20, the point-of-gaze detector 32 detects a line-of-sight vector of the subject that is set by the three-dimensional global coordinate system. The point-of-gaze detector 32 detects, as the position data on the point of gaze of the subject, position data on an intersection between the detected line-of-sight vector of the subject and the display unit 11 of the display device 10. In other words, in the embodiment, the position data on the point of gaze is the position data on the intersection between the line-of-sight vector of the subject that is set by the three-dimensional global coordinate system and the display unit 11 of the display device 10. In the embodiment, the point of gaze is a specified point on the display unit 11 that is gazed at by the subject and thus is specified. The point-of-gaze detector 32 detects the position data on the point of gaze of the subject at intervals of a sampling period that is set. For the sampling period, it is possible to set, for example, a period (for example, every 20 [msec]) of the frame synchronization signal that is output from the imaging device 21. The point-of-gaze detector 32 includes a timer that detects a time elapsing from display of the evaluation image on the display unit 11 and a counter that counts times at which the point-of-gaze detector 32 detects a point of gaze.

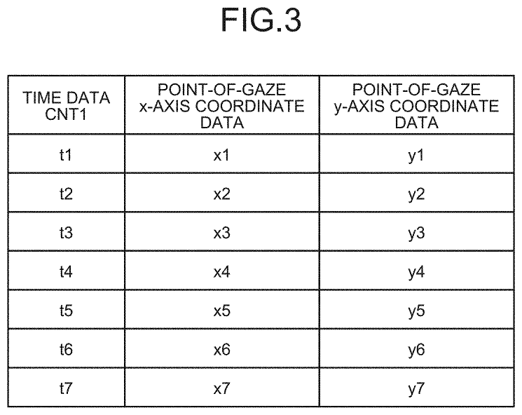

FIG. 3 is a table representing an example of point-of-gaze data that is stored in the storage 38. As illustrated in FIG. 3, the point-of-gaze detector 32 stores the number of counts of the counter (CNT1) and X-axis coordinate data and Y-axis coordinate data on the point of gaze on the display unit 11 in association with each other in the storage 38.

The determination unit 33 determines whether the point of gaze is staying based on the position data on the point of gaze and stores determination data in the storage 38. The determination unit 33 determines whether the point of gaze is staying at intervals of a period of determining that is set. The period of determining can be, for example, the same period as the period (for example, every 20 [msec]) of the frame synchronization signal that is output from the imaging device 21. In this case, the period of determining by the determination unit 33 is the same as the period of sampling by the point-of-gaze detector 32. In the embodiment, staying of the point of gaze can be a state in which the point of gaze is detected within an area with a given radius sequentially for a given number of times or more. For example, when the point of gaze is detected within an area with a radius of 100 pixels sequentially for 10 times or more, the determination unit 33 is able to determine that the point of gaze is staying. The determination data contains a time of staying in the case where it is determined that the point of gaze is staying. The time of staying can be a time from the time at which the point of gaze determined as staying is detected for the first time to the time at which the point of gaze is detected for the last time.

When the determination unit 33 determines that the point of gaze is staying, the area detector 34 detects an area in which the point of gaze is staying in the display unit 11. In the embodiment, the area of staying can be an area within a given radius (for example, a radius of 100 pixels) about the point of gaze that is detected for the first time among the sequential 10 points of gaze that are determined as staying. When the point of gaze is staying in a plurality of locations in the display unit 11 at different sets of timing, the area detector 34 is able to detect a plurality of areas of staying. The area detector 34 stores information on the area of staying, such as the point of gaze serving as the center point of the area of staying and its X-axis coordinate and the Y-axis coordinate, as area data in the storage 38.

The acquisition unit 35 acquires a feature value representing a state of display of the area of staying that is detected by the area detector 34. In the embodiment, the feature value contains at least one of luminance, contrast, sharpness, denseness and chroma. The acquisition unit 35 detects, as the contrast among the aforementioned feature values, for example, a difference between a maximum value of luminance of a white portion within the area of staying and a minimum value of luminance of a black portion. The acquisition unit 35 detects, as the sharpness, for example, an overshoot of an edge portion of the area of staying. The acquisition unit 35 detects, as the denseness, for example, how many liner portions are present in the area of staying. When a plurality of areas of staying are detected, the acquisition unit 35 acquires a feature value of each of the areas of staying. The acquisition unit 35 stores the detected feature value as feature value data in the storage 38.

Based on the result of acquisition by the acquisition unit 35, the evaluation unit 36 calculates evaluation data on the subject. The evaluation data contains data for making an evaluation on what area of the evaluation image displayed on the display unit 11 the subject is gazing at and what feature value the area has.

The input/output controller 37 acquires data from at least one of the image acquisition device 20 and the input device 50 (such as the image data on the eyeballs EB and input data). The input/output controller 37 outputs the data to at least one of the display device 10 and the output device 40.

The storage 38 stores the point-of-gaze data (refer to FIG. 3), the determination data, the data on the area of staying, the feature value data and the evaluation described above. The storage 38 stores an evaluation program for causing a computer to execute processes of detecting a position of a point of gaze of a subject on the display unit 11; displaying, on the display unit 11, an image containing an induction area that tends to induce visual hallucinations in a subject; based on position data on the point of gaze, determining whether the point of gaze is staying; when it is determined that the point of gaze is staying, detecting an area of staying in which the point of gaze is staying in the display unit 11; acquiring a feature value representing a state of display of the area of staying; and, based on the acquired feature value, calculating evaluation data on the subject.

### Evaluation Method

The evaluation method according to the embodiment will be described next. In the evaluation method according to the embodiment, by using the evaluation device 100 described above, an evaluation on cognitive dysfunction and brain dysfunction of a subject is made. In the embodiment, for example, the case where an evaluation is made on dementia with Lewy bodies serving as cognitive dysfunction and brain dysfunction is described.

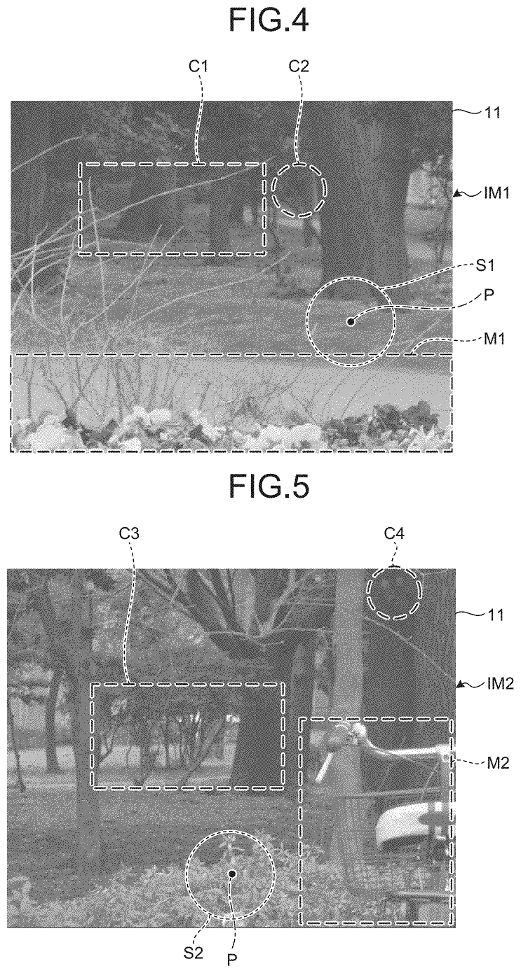

FIG. 4 is a diagram illustrating an example of the evaluation image that is displayed on the display unit 11. As illustrated in FIG. 4, the display controller 31 causes the display unit 11 to display a photographic image as an evaluation image IM1. The evaluation image IM1 is a photographic image on which flowers are on a lower side in the drawing and trees are on an upper side in the drawing. The evaluation image IM1 is a photographic image obtained by capturing an image of a scenery in which the flowers are present on the front side in the depth direction and the trees are present on the back side in the depth direction, with the flowers serving as a position of a focal point. Thus, in the evaluation image IM1, the sharpness, luminance, and contrast of the area with the flowers are higher than those of surrounding areas. In the embodiment, the evaluation image IM1 contains the area with the flowers serving as the subject as a target area Ml. The target area M1 is an area serving as a target that the subject is caused to gaze at.

A symptom that visual hallucinations are induced is known as one of symptoms of dementia with Lewy bodies. In the case where a visual hallucination is induced in a subject suffering from dementia with Lewy bodies when the subject gazes at an image, the subject tends to gaze at an area serving as a subject of the visual hallucination. When an evaluation on such dementia with Lewy bodies is made, an image containing an area that induces visual hallucinations is shown to a subject.

In the evaluation image IM1 of the embodiment, the area of the trees on the back side is different from the target area M1 in the following aspects.

1. The focus is on the flowers and the trees are out of focus. In other words, the area of the trees has a lower sharpness than that of the target area M1.

2. The area of the trees has a lower luminance than that of (is darker than) the target area M1.

3. The area of the trees has a lower contrast than that of the target area M1.

In the case where the evaluation image IM1 is a color image, an image in which the area of the trees has a lower chroma than that of the target area M1 is used.

As a result, in the evaluation image IM1, the area of the trees contains areas that tend to induce visual hallucinations in a subject suffering from dementia with Lewy bodies. For example, an area C1 that is part of the area of the trees serves as an area that induces visual hallucinations of the figure of a person, and the like, in a subject suffering from dementia with Lewy bodies. The area C1 is referred to as an induction area C1 below. For example, an area C2 that is part of the area of the trees serves as an area that induces visual hallucinations of the face of a person, and the like, in a subject suffering from dementia with Lewy bodies. The area C2 is referred to as an induction area C2 below.

On the other hand, the induction areas C1 and C2 are areas that are merely part of the area of the trees whose sharpness, luminance and contrast (or chroma) are low to a subject not suffering from dementia with Lewy bodies and that tend not to induce visual hallucinations of the figure of a person, the face of a person, etc. Thus, in the case where the subject is not suffering from dementia with Lewy bodies, when the evaluation image IM1 is displayed on the display unit 11, the subject is highly likely to gaze at the target area M1 whose sharpness, luminance and contrast are high.

In the embodiment, for example, it is possible to use an image with which a significant difference is shown between a subject not suffering from dementia with Lewy bodies and a subject suffering from dementia with Lewy bodies after evaluations are made on a plurality of subjects. In other words, it is possible to use an image containing the target area M1 that a subject not suffering from dementia with Lewy bodies more tends to gaze at and the induction areas C1 and C2 that a subject not suffering from dementia with Lewy bodies more tends not to gaze at and a subject suffering from dementia with Lewy bodies more tends to gaze at.

Furthermore, using the above-described photographic image as the evaluation image IM1 more assuredly enables a subject not suffering from dementia with Lewy bodies to gaze at the subject in the target area M1 on the front side. Furthermore, the induction areas C1 and C2 that cause a less sense of discomfort and that is natural to a subject not suffering from dementia with Lewy bodies are contained in the evaluation image IM1. Accordingly, a subject not suffering from dementia with Lewy bodies is inhibited from gazing at the induction areas C1 and C2.

During the period in which the evaluation image IM1 is displayed, the point-of-gaze detector 32 detects position data on a point of gaze P of the subject at intervals of the period of sampling that is set (for example, 20 [msec]). The determination unit 33 determines whether the point of gaze is staying based on the position data on the point of gaze P. The determination unit 33 is able to determine that the point of gaze P is staying, for example, when the point of gaze P is detected in an area with a radius of 100 pixels or less sequentially for 10 times or more.

When it is determined that the point of gaze is staying, the area detector 34 detects the area in which the point of gaze P is staying in the display unit 11. The area detector 34 is able to detect, as the area of staying, for example, an area with a radius of 100 pixels or less about the point of gaze that is detected for the first time among sequential 10 points of gaze or more that are determined as staying. FIG. 4 illustrates the state where an area of staying S1 is detected.

The acquisition unit 35 acquires a feature value representing a state of display of the area of staying S1 that is detected by the area detector 34. The acquisition unit 35 detects, as the feature value, at least one of luminance, contrast, sharpness, denseness and chroma. When a plurality of areas of staying are detected, the acquisition unit 35 detects feature data on each of the areas of staying individually. The acquisition unit 35 stores the detected feature value as feature value data in the storage 38.

The evaluation unit 36 calculates an evaluation value based on the acquired feature value and calculates evaluation data based on the feature value. When a plurality of areas of staying are detected, the evaluation unit 36 calculates evaluation values on the areas of staying. When calculating evaluation values on the areas of staying, the evaluation unit 36 is able to calculate evaluation values sequentially from an area of staying where the time during which the point of gaze is staying is long.

For example, on the area of staying with the longest time of staying, the evaluation unit 36 determines whether the denseness is at a given value or higher based on the feature value data and, when the denseness is at the given value or higher, sets a coefficient K1. The evaluation unit 36 determines whether the contrast is at a given value or higher and, when the contrast is at the given value or higher, sets a coefficient K2. The evaluation unit 36 determines whether the sharpness is at a given value or higher and, when the sharpness is at the given value or higher, sets a coefficient K3. The evaluation unit 36 determines whether the luminance is at a given value or higher and, when the contrast is at the given value or higher, sets a coefficient K4. The evaluation unit 36 determines whether the chroma is at a given value or higher and, when the chroma is at the given value or higher, sets a coefficient K5. The evaluation unit 36 calculates an evaluation value A1 based on the coefficients that are set. When the time of staying is T1, the evaluation value A1 is represented by

A1=(K1+K2+K3+K4+K5)·T1

When there is an area of staying with the second longest time of staying, as described above, the evaluation unit 36 sets, for the area of staying, a coefficient K6 for denseness, a coefficient K7 for contrast, a coefficient K8 for sharpness, a coefficient K9 for luminance, and a coefficient K10 for chroma and calculates an evaluation value A2. When the time of staying is T2, the evaluation value A2 is represented as follows.

A2=(K6+K7+K8+K9+K10)·T2

When there is an area of staying with the third longest time of staying, as described above, the evaluation unit 36 sets, for the area of staying, a coefficient K11 for denseness, a coefficient K12 for contrast, a coefficient K13 for sharpness, a coefficient K14 for luminance, and a coefficient K15 for chroma and calculates an evaluation value A3. When the time of staying is T3, the evaluation value A3 is represented as follows.

A3=(K11+K12+K13+K14+K15)·T3

As described above, the evaluation unit 36 is able to calculate evaluation values to an evaluation value AN of an area of staying with the N-th longest time of staying. It is possible to set the coefficients K1 to K15 described above, for example, according to the features of the induction areas C1 and C2.

The evaluation unit 36 then calculates a final evaluation value ANS based on the evaluation values A1 to AN described above. The evaluation value ANS is represented by

ANS=α1·A1+α2·A2+α3·A3+ . . . +αN·AN

where α1, α2, α3, . . . , αN are constants. For these constants, α1>α2>α3, . . . , >αN may be set. This makes it possible to obtain an evaluation value ANS obtained by weighting the evaluation values on the areas of staying with long times of staying.

When ANS is equal to or larger than a given value, it is considered that the portion that the subject gazes at is a portion whose denseness, contrast and sharpness in the evaluation image are high, that is, a part that an able-bodied person is highly likely to look at. When the ANS is smaller than the given value, it is considered that the portion gazed at by the subject is a portion whose denseness, contrast and sharpness are low in the evaluation image and that thus tends to cause visual hallucinations, that is, a portion that a patient suffering from dementia with Lewy bodies is highly likely to look at.

Accordingly, by determining whether the evaluation value ANS is equal to or larger than the given value, the evaluation unit 36 is able to calculate evaluation data. For example, when the evaluation value ANS is equal to or larger than the given value, it is possible to evaluate that the subject is less likely to be suffering from dementia with Lewy bodies. When the evaluation value ANS is smaller than the given value, it is possible to evaluate that the subject is highly likely to be suffering from dementia with Lewy bodies.

The evaluation unit 36 is able to store the value of the evaluation value ANS in the storage 38. For example, the evaluation values ANS on the same subject may be stored cumulatively and an evaluation may be made in comparison with an evaluation value in the past. For example, when the evaluation value ANS is a higher value than the evaluation value in the past, it is possible to evaluate that the brain function has improved compared to the previous evaluation. In the case where the cumulative value of evaluation values ANS gradually increases, or the like, it is possible to evaluate that the brain function is gradually improving.

In the embodiment, when the evaluation unit 36 outputs evaluation data, the input/output controller 37 is able to cause the output device 40 to output, according to the evaluation data, for example, character data of “The subject seems to be less likely to be suffering from dementia with Lewy bodies.”, character data “The subject seems to be highly likely to be suffering from dementia with Lewy bodies.”, or the like. When the evaluation value ANS on the same subject is higher than the evaluation value ANS in the past, the input/output controller 37 is able to cause the output device 40 to output character data of “The brain function has improved”, or the like.

FIG. 5 is a diagram illustrating another example of the evaluation image that is displayed on the display unit 11. As illustrated in FIG. 5, the display controller 31 causes the display unit 11 to display a photographic image as an evaluation image IM2. The evaluation image IM2 is a photographic image on which a bicycle is on a lower right side in the drawing and trees are on an upper side in the drawing. The evaluation image IM2 is a photographic image obtained by capturing an image of a scenery in which the bicycle is present on the front side in the depth direction and the trees are present on the back side in the depth direction, with the bicycle serving as a position of a focal point. Thus, in the evaluation image IM2, the sharpness, luminance, and contrast of the area with the bicycle are higher than those of surrounding areas. In the embodiment, the evaluation image IM2 contains the area with the bicycle serving as a subject as a target area M2. The target area M2 is an area serving as a target that the subject is caused to gaze at.

On the other hand, in the evaluation image IM2, the area of the trees on the back side is different from the target area M2 in the following aspects.

4. The focus is on the bicycle and the trees are out of focus. In other words, the area of the trees has a lower sharpness than that of the target area M2.

5. The area of the trees has a lower luminance than that of (is darker than) the target area M2.

6. The area of the trees has a lower contrast than that of the target area M2.

In the case where the evaluation image IM2 is a color image, an image in which the area of the trees has a lower chroma than that of the target area M2 is used.

As a result, an area that tends to induce visual hallucinations in a subject suffering from dementia with Lewy bodies is present in the evaluation image IM2. For example, an area C3 that is part of the area of the trees serves as an area that induces a visual hallucination of the figure of a person in a subject suffering from dementia with Lewy bodies. The area C3 is referred to as an induction area C3 below. An area C4 that is part of the area of the trees serves as an area that induces a visual hallucination of the face of a person in a subject suffering from dementia with Lewy bodies. The area C4 is referred to as an induction area C4 below.

During the period in which the evaluation image IM2 is displayed, the point-of-gaze detector 32 detects position data on the point of gaze P of the subject at intervals of a sampling period that is set (for example, 20 [msec]). Based on the position data on the point of gaze P, the determination unit 33 determines whether the point of gaze is staying. When it is determined that the point of gaze is staying, the area detector 34 detects an area in which the point of gaze P is staying in the display unit 11. FIG. 5 illustrates a state where an area of staying S2 is detected. The acquisition unit 35 acquires a feature value representing a state of display of the area of staying S2 that is detected by the area detector 34. The acquisition unit 35 stores the detected feature value as feature value data in the storage 38.

The display controller 31 is able to display the evaluation image IM2 illustrated in FIG. 5 on the display unit 11 for a given time, for example, after displaying the evaluation image IM1 illustrated in FIG. 4 for a given time. As described above, displaying multiple types of the evaluation images IM1 and IM2 on the display unit 11 enables accurate evaluation on the subject.

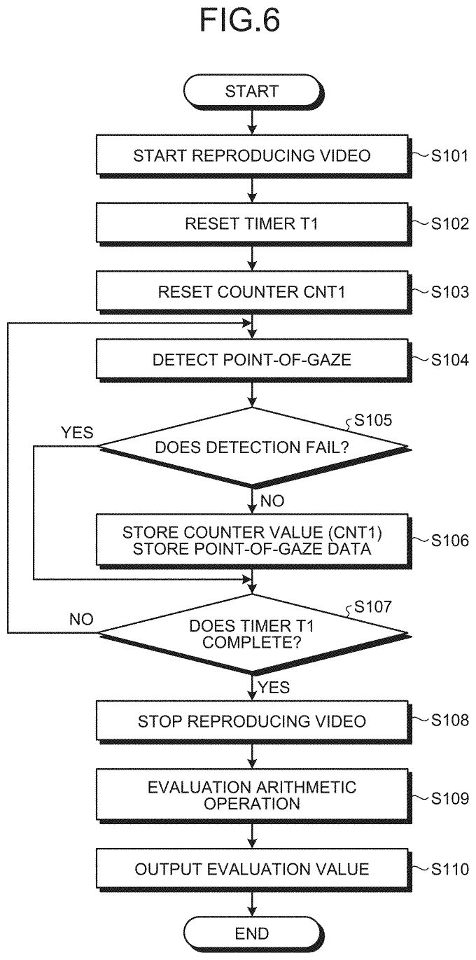

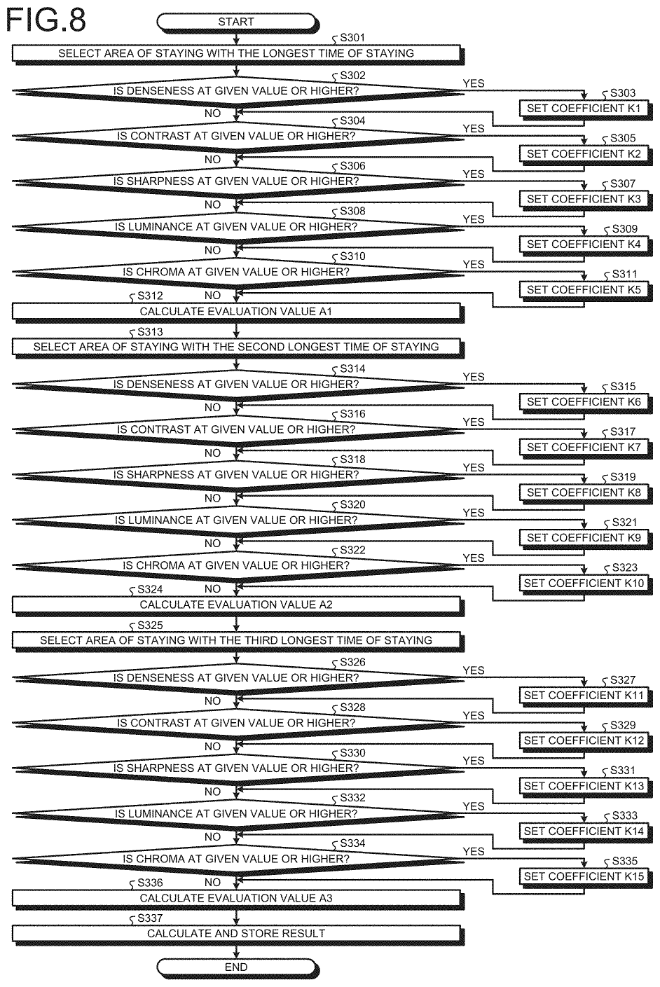

An example of the evaluation method according to the embodiment will be described with reference to FIGS. 6 and 8. FIG. 6 is a flowchart illustrating the example of the evaluation method according to the embodiment. The following example will be described, exemplifying the case where the evaluation image IM1 is reproduced as a video (evaluation video). As illustrated in FIG. 6, the display controller 31 starts reproduction of an evaluation image (display of the evaluation image IM1) (step S101). The evaluation image IM1 may be displayed in the interval between displays of images for making other evaluations on a subject. After reproduction of the evaluation image IM1 is started, the timer T1 is reset (step S102) and the count value CNT1 is reset (step S103).

The point-of-gaze detector 32 detects position data on the point of gaze of the subject on the display unit 11 at intervals of the sampling period that is set (for example, 20 [msec]) in the state where the evaluation image IM1 displayed on the display unit 11 is being shown to the subject (step S104). When position data is not detected (YES at step S105), the process at and after step S107 is performed. When position data is detected (NO at step S105), the point-of-gaze detector 32 counts the time at which the point of gaze is detected by the counter and stores the count value CNT1 in the storage 38 and stores an X-axis coordinate and a Y-axis coordinate of the detected point of gaze in association with the count value CNT1 in the storage 38 (S106).

Thereafter, based on a detection result of the detection timer T1, the point-of-gaze detector 32 determines whether a time at which reproduction of the evaluation image completes is reached (step S107). When it is determined that the time at which reproduction of the evaluation image completes is not reached (NO at step S107), the process at and after step S104 is performed repeatedly.

When the point-of-gaze detector 32 determines that the time at which reproduction of the evaluation image completes is reached (YES at step S107), the display controller 31 stops reproduction of the video relating to an instruction display operation (step S108). After reproduction of the video is stopped, the determination unit 33, the area detector 34, the acquisition unit 35 and the evaluation unit 36 performs evaluation arithmetic operations for calculating an evaluation value ANS (step S109). The evaluation arithmetic operations will be described below. The evaluation unit 36 calculates evaluation data based on the evaluation value ANS obtained by evaluation arithmetic operations. Thereafter, the input/output controller 37 outputs the evaluation data that is calculated by the evaluation unit 36 (step S110).

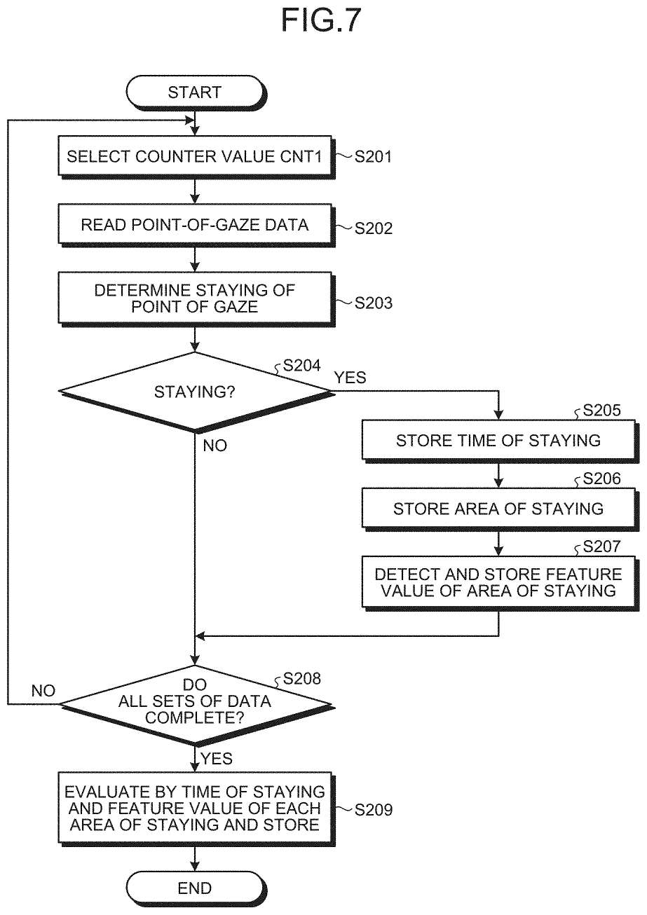

FIG. 7 is a flowchart illustrating an example of the evaluation arithmetic operations according to the embodiment. As illustrated in FIG. 7, the determination unit 33 sets a count value CNT1 representing a time at which a point of gaze is detected (step S201) and reads point-of-gaze data corresponding to the count value CNT1 (X-axis coordinate data and Y-axis coordinate data) from the storage 38 (step S202). Based in the point-of-gaze data that is read, the determination unit 33 detects whether the point of gaze is staying (step S203).

As a result of detection at step S203, when it is determined that the point of gaze is not staying (NO at step S204), the process at and after step S208 to be described below is performed. When it is determined that the point of gaze is staying (YES at step S204), the determination unit 33 calculates a time of staying during which the point of gaze is staying and stores the time of staying in the storage 38 (step S205). The area detector 34 detects an area of staying and stores area data on the area of staying in the storage 38 (step S206). Thereafter, the acquisition unit 35 acquires feature values of the area of staying (luminance, contrast, sharpness, denseness, and chroma) and stores the acquired feature values in the storage 38 (step S207).

Thereafter, the determination unit 33 determines whether the above-described process on all the sets of point-of-gaze data completes (step S208) and, when it is determined that the process does not complete (NO at step S208), repeats the process at and after step S201. When the determination unit 33 determines that the process completes (YES at step S208), the evaluation unit 36 calculates an evaluation value ANS based on the acquired feature values and stores evaluation data that is calculated in the storage 38 (step S209).

FIG. 8 is a flowchart specifically illustrating the process of step S209. Description will be given, exemplifying the case where three areas of staying or more are detected. As illustrated in FIG. 8, the evaluation unit 36 selects an area of staying with the longest time of staying among the detected areas of staying (step S301). The evaluation unit 36 then determines whether the denseness in the selected area of staying is at the given value or higher (step S302). When it is determined that the denseness is at the given value or higher (YES at step S302), the evaluation unit 36 sets a coefficient K1 for denseness (step S303) and moves to step S304. When it is determined that the denseness is not at the given value or higher (NO at step S302), the evaluation unit 36 moves to step S304 without setting a coefficient K1 for denseness.

The evaluation unit 36 then determines whether the contrast is at the given value or higher (step S304). When it is determined that the contrast is at the given value or higher (YES at step S304), the evaluation unit 36 sets a coefficient K2 for contrast (step S305) and moves to step S306. When it is determined that the contrast is not at the given value or higher (NO at step S304), the evaluation unit 36 moves to step S306 without setting a coefficient K2 for contrast.

The evaluation unit 36 then determines whether the sharpness is at the given value or higher (step S306). When it is determined that the sharpness is at the given value or higher (YES at step S306), the evaluation unit 36 sets a coefficient K3 for sharpness (step S307) and moves to step S308. When it is determined that the sharpness is not at the given value or higher (NO at step S306), the evaluation unit 36 moves to step S308 without setting a coefficient K3 for sharpness.

The evaluation unit 36 then determines whether the luminance is at the given value or higher (step S308). When it is determined that the luminance is at the given value or higher (YES at step S308), the evaluation unit 36 sets a coefficient K4 for luminance (step S309) and moves to step S310. When it is determined that the luminance is not at the given value or higher (NO at step S308), the evaluation unit 36 moves to step S310 without setting a coefficient K4 for luminance.

The evaluation unit 36 then determines whether the chroma is at the given value or higher (step S310). When it is determined that the chroma is at the given value or higher (YES at step S310), the evaluation unit 36 sets a coefficient K5 for chroma (step S311) and moves to step S312. When it is determined that the chroma is not at the given value or higher (NO at step S310), the evaluation unit 36 moves to step S312 without setting a coefficient K5 for chroma.

Based on the coefficients K1 to K5 that are set, the evaluation unit 36 calculates an evaluation value A1 on the area of staying with the longest time of staying (step S312).

The evaluation unit 36 then selects an area of staying with the second longest time of staying (step S313) and, as described above, sets a coefficient K6 for denseness, a coefficient K7 for contrast, a coefficient K8 for sharpness, a coefficient K9 for luminance, and a coefficient K10 for chroma (steps S314 to step S323). Based on the coefficients K6 to K10 that are set, the evaluation unit 36 calculates an evaluation value A2 on the area of staying with the second longest time of staying (step S324).

The evaluation unit 36 selects an area of staying with the third longest time of staying (step S325) and, as described above, sets a coefficient K11 for denseness, a coefficient K12 for contrast, a coefficient K13 for sharpness, a coefficient K14 for luminance, and a coefficient K15 for chroma (steps S326 to step S335). Based on the coefficients K11 to K15 that are set, the evaluation unit 36 calculates an evaluation value A3 on the area of staying with the third longest time of staying (step S336).

Thereafter, based on the calculated evaluation values A1 to A3 and the times of staying, the evaluation unit 36 calculates a final evaluation value ANS (step S337).

When a single area of staying is detected, the process from step S313 to step S336 is not performed. When two areas of staying are detected, the process from step S325 to step S336 is not performed. When four areas of staying or more are detected, as described above, a coefficient for denseness, a coefficient for contrast, a coefficient for sharpness, a coefficient for luminance, and a coefficient for chroma may be set and a final ANS may be set using the result of the setting.

As described above, the evaluation device 100 according to the embodiment includes the display unit 11; the point-of-gaze detector 32 that detects a position of a point of gaze of a subject on the display unit 11; the display controller 31 that displays, on the display unit 11, an image containing an induction area that tends to induce visual hallucinations in the subject; the determination unit 33 that, based on position data on the point of gaze, determine whether the point of gaze is staying; the area detector 34 that, when the determination unit 33 determines that the point of gaze is staying, calculates an area of staying in which the point of gaze is staying in the display unit 11; the acquisition unit 35 that acquires a feature value representing a state of display of the area of staying calculated by the area detector 34; and the evaluation unit 36 that, based on the feature value that is acquired by the acquisition unit 35, calculates evaluation data on the subject.

The evaluation method according to the embodiment includes detecting a position of a point of gaze of a subject on the display unit 11; displaying, on the display unit 11, an image containing an induction area that tends to induce visual hallucinations in the subject; based on position data on the point of gaze, determining whether the point of gaze is staying; when it is determined that the point of gaze is staying, detecting an area of staying in which the point of gaze is staying in the display unit 11; acquiring a feature value representing a state of display of the area of staying; and based on the feature value that is acquired, calculating evaluation data on the subject.

The evaluation program according to the embodiment causes a computer to execute processes of detecting a position of a point of gaze of a subject on the display unit 11; displaying, on the display unit 11, an image containing an induction area that tends to induce visual hallucinations in the subject; based on position data on the point of gaze, determining whether the point of gaze is staying; when it is determined that the point of gaze is staying, detecting an area of staying in which the point of gaze is staying in the display unit 11; acquiring a feature value representing a state of display of the area of staying; and based on the feature value that is acquired, calculating evaluation data on the subject.

In the case where the subject is not suffering from dementia with Lewy bodies, when the evaluation image IM1 is displayed on the display unit 11, the subject is highly likely to gaze at the target area M1 whose feature value is high. In the case where the subject is suffering from dementia with Lewy bodies, when the evaluation image IM1 is displayed on the display unit 11, the subject is highly likely to gaze at the induction area C1 or C2 that induces visual hallucinations. According to the above-described configuration, because a subject is caused to gaze at the evaluation image IM1 in which the area that tends to induce visual hallucinations in a subject suffering from dementia with Lewy bodies is present, it is possible to make an evaluation not depending on the subjective view of the subject. It is also possible to reduce the effect of contingency because it is possible to calculate evaluation data based on the point-of-gaze data on the subject.

In the evaluation device 100 according to the embodiment, the feature value contains at least one of luminance, contrast, sharpness, denseness, and chroma. This makes it possible to evaluate a subject accurately.

In the evaluation device 100 according to the embodiment, the determination unit 33 calculates a time of staying during which the point of gaze stays for each area of staying and, when a plurality of areas of staying are detected, the evaluation unit 36 calculates evaluation data obtained by weighting areas of staying with long times of staying. Accordingly, the evaluation most reflects the feature value of the area at which the subject gazes for the longest time and thus it is possible to accurately evaluate the subject.

In the evaluation device 100 according to the embodiment, the display unit 11 displays the evaluation image IM1 containing, in the position different from those of the induction areas C1 and C2, the target area M1 serving as the target that the subject is caused to gaze at and the feature values of the induction areas C1 and C2 are lower than that of the target area M1. Accordingly, the area that tends to cause visual hallucinations in a subject suffering from dementia with Lewy bodies can be contained efficiently.

In the evaluation device 100 according to the embodiment, the display unit 11 displays the evaluation image IM1 containing, in the position different from those of the induction areas C1 and C2, the target area M1 serving as the target that the subject is caused to gaze at and the feature value of the target area M1 is higher than that of the surroundings. Accordingly, a subject not suffering from dementia with Lewy bodies is to gaze at the target area M1 and thus it is possible to make an accurate evaluation.

In the evaluation device 100 according to the embodiment, the evaluation image IM1 is a photographic image in which the subject in the target area M1 is present on the front side in the depth direction and the subject in the induction areas C1 and C2 is present on the back side in the depth direction and that is captured with the subject in the target area M1 serving as a position of a focal point. This more assuredly enables a subject not suffering from dementia with Lewy bodies to gaze at the subject in the target area M1 on the front side. Furthermore, the induction areas C1 and C2 that are less artificial and natural can be contained in the evaluation image IM1. Thus, it is possible to inhibit a subject not suffering from dementia with Lewy bodies from gazing at the induction areas C1 and C2 and make an accurate evaluation.

In the evaluation device 100 according to the embodiment, images with which a significant difference is shown between a subject not suffering from dementia with Lewy bodies and a subject suffering from dementia with Lewy bodies as a result of evaluation by the evaluation unit 36 are used as the evaluation images IM1 and IM2. Accordingly, it is possible to accurately make an evaluation on the possibility that a subject would suffer from dementia with Lewy bodies.

The technical scope of the disclosure is not limited to the embodiment described above and changes can be added as appropriate without departing from the purpose of the disclosure. For example, the above-described embodiment has been described, exemplifying the case where the induction areas C1 to C4 that induce visual hallucinations of the figure of a person or the face of a person are contained in the evaluation images IM1 and IM2; however, embodiments are not limited thereto. For example, an induction area that induces a visual hallucination of an animal other than human, such as a small animal or an insect, may be contained in the evaluation image.

In the above-described embodiment, the case where a photographic image is displayed as an evaluation image has been described; however, the evaluation image is not limited to a photographic image and a drawn or created image may be used.

In the above-described embodiment, description is given, exemplifying the case where an evaluation value is calculated on part of areas of staying with long times of staying that are detected; however, embodiments are not limited thereto and evaluation values may be calculated on all areas of staying.

According to the disclosure, it is possible to provide an evaluation device, and evaluation method, and an evaluation program that enable accurate evaluation on cognitive dysfunction and brain dysfunction.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. An evaluation device comprising:
a display unit;
a point-of-gaze detector configured to detect a position of a point of gaze of a subject on the display unit;
a display controller configured to display, on the display unit, an image containing an induction area that tends to induce visual hallucinations in the subject;
a determination unit configured to, based on position data on the point of gaze, determine whether the point of gaze is staying;
an area detector configured to, when the determination unit determines that the point of gaze is staying, calculate an area of staying in which the point of gaze is staying in the display unit;
an acquisition unit configured to acquire a feature value representing a state of display of the area of staying that is calculated by the area detector; and
an evaluation unit configured to, based on the feature value that is acquired by the acquisition unit, calculate evaluation data on the subject.

2. The evaluation device according to claim 1, wherein the feature value contains at least one of luminance, contrast, sharpness, denseness, and chroma.

3. The evaluation device according to claim 1, wherein the determination unit is configured to calculate a time of staying during which the point of gaze stays for each area of staying, and
the evaluation unit is configured to, when a plurality of the areas of staying are detected, calculate the evaluation data obtained by weighting the area of staying with the time of staying that is long.

4. The evaluation device according to claim 1, wherein the display unit is configured to display an image containing, in a position different from that of the induction area, a target area serving as a target that the subject is caused to gaze at, and
the feature value of the induction area is lower than that of the target area.

5. The evaluation device according to claim 1, wherein the display unit is configured to display an image containing, in a position different from that of the induction area, a target area serving as a target that the subject is caused to gaze at, and
the feature value of the target area is higher than that of surroundings.

6. The evaluation device according to claim 1, wherein the image is a photographic image in which a subject in the target area is present on a front side in a depth direction and a subject in the induction area is present on a back side in the depth direction and that is captured with the subject in the target area serving as a position of a focal point or an image that is drawn and produced.

7. The evaluation device according to claim 1, wherein an image with which a significant difference is shown between a subject not suffering from dementia with Lewy bodies and a subject suffering from dementia with Lewy bodies as a result of evaluation by the evaluation unit is used as the image.

8. An evaluation method comprising:
detecting a position of a point of gaze of a subject on a display unit;
displaying, on the display unit, an image containing an induction area that tends to induce visual hallucinations in the subject;
based on position data on the point of gaze, determining whether the point of gaze is staying;
when it is determined that the point of gaze is staying, detecting an area of staying in which the point of gaze is staying in the display unit;
acquiring a feature value representing a state of display of the area of staying; and
based on the feature value that is acquired, calculating evaluation data on the subject.

9. A non-transitory computer readable recording medium storing therein an evaluation program for causing a computer to execute processes of
detecting a position of a point of gaze of a subject on a display unit;
displaying, on the display unit, an image containing an induction area that tends to induce visual hallucinations in the subject;
based on position data on the point of gaze, determining whether the point of gaze is staying;
when it is determined that the point of gaze is staying, detecting an area of staying in which the point of gaze is staying in the display unit;
acquiring a feature value representing a state of display of the area of staying; and
based on the feature value that is acquired, calculating evaluation data on the subject.
